(19) United States
(12) Patent Application Publication
(10) Pub. No.: US 2018/0094058 A1
(43) Pub. Date: Apr. 5, 2018

(54) COMPOSITIONS AND METHODS FOR PREVENTING TUMOR GROWTH AND TREATING CANCER BY TARGETING LECTIN GALACTOSIDE-BINDING SOLUBLE 3 BINDING PROTEIN

(71) Applicant: BioXcel Corporation, Branford, CT (US)

(72) Inventors: Himani SHARMA, Pune (IN); Krishnan Nandabalan, Guilford, CT (US)

(21) Appl. No.: 15/567,251

(22) PCT Filed: Apr. 18, 2016
(86) PCT No.: PCT/US16/28070

§ 371 Date: Oct. 17, 2017

Related U.S. Application Data
(60) Provisional application No. 62/148,933, filed on Apr. 17, 2015.

(51) Int. Cl. C07K 16/28

(52) U.S. Cl. CPC C07K 16/2851; A61K 2039/507; C07K 16/2818

(57) ABSTRACT

The present invention discloses a method of treating, preventing or ameliorating tumor growth by immune response modulation via targeting LGALS3BP-CD33 related Siglec pathway using antibody or antibody-drug conjugate therapy. The present invention also provide use of anti-LGALS3BP antibody in combination with an immune checkpoint inhibitor for enhancing, increasing, promoting, expressing, modulating desirable immune response for prevention and treatment of tumors and metastases thereof. Also provides combination therapy with an immune checkpoint inhibitor.

### CROSS REFERENCE TO RELATED APPLICATION

This application incorporates U.S. Provisional Application Ser. No. 62/148,933 filed Apr. 17, 2015 in its entirety for all purposes.

### FIELD OF THE INVENTION

The present invention is in the field of biopharmaceuticals and more specifically relates to an identification of a novel approach for targeting tumor associated immunomodulatory ligands using antibodies. In particular, the invention is directed to combinations of antibodies targeting LGALS3BP and an immune checkpoint target (such as PDL1, PD1 or CTLA4 ). Methods of using such therapeutic antibodies and compositions containing them are provided

### BACKGROUND OF THE INVENTION

Cancer is not fully understood on a molecular level and remains a leading cause of death worldwide. The development of human cancer is a multistep process characterized by the accumulation of progressive genetic alterations that confer to tumor cells the ability to survive, proliferate, and metastasize. Once they have expanded to a critical level, tumor cells find a way to promote new vasculature development, a process known as tumor angiogenesis in order to progress and metastasize. Tumor cells are also known to successfully evade immune surveillance mechanisms. LGALS3BP is a ligand of the lactose-specific S-type lectin, galectin-3 (formerly Mac-2) and is composed of 90-kDa subunits. It is up-regulated in many cancers and has been implicated in immune response associated with natural killer (NK) and lymphokine-activated killer (LAK) cell cytotoxicity.

H. Läubli et, al. (The Journal of Biological Chemistry Vol. 289, No. 48, pp. 33481-33491, Nov. 28, 2014) provides a superficial disclosure that the LGALS3BP could promote immune evasion by inhibiting immune cell activation through engagement of Siglecs and defines LGALS3BP-Siglec interactions as potential novel target to interfere with cancer progression and reactivate the immune system against carcinomas.

U.S. Pat. No. 8,679,495 assigned to Mediapharma S.R.L., discloses the use of anti-90 K antibody i.e. monoclonal antibody SP-2 for prevention and treatment of tumors and their metastases. This patent emphasizes that monoclonal antibody SP-2 inhibits the pro-adhesive function of 90K. It also provides the combination of monoclonal antibody SP-2 and anti-tumor agents. However, this publication is silent about the usage of combination of immune checkpoint inhibitors and anti-LGALS3BP antibody as the promising immunotherapy for the cancer patients.

Elevated serum or tissue levels of lectin galactoside binding soluble 3 binding protein (LGALS3BP) are associated with short survival and development of metastasis in a variety of human cancers. However, the role of LGALS3BP, particularly in the context of tumor-host relationships was not studied in detail in the past and also there is no hint in the prior art about the combinations of the present invention.

Immune checkpoint inhibitors such as Programmed Cell Death 1 (PD-1) is a cell surface signaling receptor that plays a critical role in the regulation of T cell activation and tolerance. PD-1 is primarily expressed on activated T cells, B cells, and myeloid cells. PD-1 is highly expressed on tumor-infiltrating lymphocytes, and its ligands such as PD-L1 are up-regulated on the cell surface of many different tumors. It has been shown that inhibition of the PD-1/PD-L1 interaction mediates potent antitumor activity in preclinical models (U.S. Pat. Nos. 8,008,449 and 7,943,743). Cytotoxic T-lymphocyte antigen 4 (CTLA-4) as an immune checkpoint, downregulates the immune system. It is found on the surface of T-cells and involved in the maintenance of T cell homeostasis. U.S. Pat. No. 7,452,535 discloses a method of treating cancer by administration of anti-CTLA4 antibodies. These patents do not provide any teachings to control the tumor growth by modulating the immune response via targeting both LGALS3BP-CD33 related Siglec pathway and immune checkpoint such as PD-1, PD-L1, PD-L2 or CTLA4 or via combination of both anti LGALS3BP antibody and an immune checkpoint inhibitor.

Despite advances in the field, however, there remains a need for improved methods and compositions for treating cancer

### SUMMARY OF THE INVENTION

Unexpectedly, the present inventors have identified that tumor growth could be controlled by modulating the immune response via targeting LGALS3BP-CD33 related Siglec pathway and this could be achieved by using a specific antibody against LGALS3BP. The antibody will activate an immune response which helps in delaying the growth of human tumors. The present inventors surprisingly found that the tumor growth could be effectively controlled by modulating the immune response via targeting both LGALS3BP-CD33 related Siglec pathway and immune checkpoint such as PD-1, PDL-1 or CTLA4 and this could be achieved by using combination of anti-LGALS3BP antibody and an immune checkpoint inhibitor.

Particularly for the combination therapy, it has been invented by the present inventors that the combination of an anti-LGALS3BP antibody and an immune checkpoint inhibitor may enhance or prolong an anti-tumor response in a subject. Further, the administration of an anti-LGALS3BP antibody with an immune checkpoint inhibitor may enhance or prolong the effects of the immune checkpoint inhibitor, enable a subject to respond to an immune checkpoint inhibitor, or enable the reduction of the toxicity or the dose of an immune checkpoint inhibitor.

It is a principal object of the present invention to provide a method of treating cancers associated with increased level of Lectin galactoside-binding soluble 3 binding protein (LGALS3BP). It also provides methods of treating tumors by modulating the immune response via LGALS3BP-CD33 related Siglec pathway.

In yet another aspect, the present invention discloses a method of enhancing, increasing, promoting, expressing, modulating desirable immune response in a subject, comprising administering an antibody(s) targeting tumors associated with increased levels of Lectin galactoside-binding soluble 3 binding protein (LGALS3BP) in an amount to enhance, increase, promote, express, modulate immune response in the subject, wherein the subject has been diagnosed for tumor.

In yet another aspect, the present invention discloses use of an anti-LGALS3BP monoclonal antibody for enhancing, increasing, promoting, expressing, modulating desirable immune response for the prevention and treatment of tumors/cancers and metastases thereof.

In a preferred aspect, the present invention provides method of enhancing, increasing, promoting, expressing, modulating desirable immune response in a subject, comprising administering a therapeutically effective amount of one or more monoclonal antibodies that bind and neutralize both LGALS3BP and an immune checkpoint target to enhance, increase, promote, express, modulate immune response(s) in the subject, wherein said subject has been diagnosed for tumor associated with increased/altered levels of LGALS3BP and/or immune checkpoint(s).

In one another aspect, the present invention discloses a pharmaceutical composition comprising one or more monoclonal antibodies that bind and neutralize LGALS3BP in combination with one or more immune checkpoint inhibitors, along with optional anti-tumor agent(s) and one or more pharmaceutically acceptable excipients and/or adjuvants. Immune checkpoint inhibitors mentioned herein include molecule or antibody that target the immune checkpoint.

The anti-tumor agent may be selected from the group consisting of an antibody, an antimetabolite, a vinca alkaloid, a taxane, an anthracycline, a platin derivative, a small molecule, a kinase inhibitor, an alkylating agent, a mTOR inhibitor. Examples of anti-tumor agents include but not limited: docetaxel, paclitaxel, doxorubicin, farmorubicin, cyclophosphamide, 5-fluorouracil, vinorelbine, cisplatin, carboplatin, trastuzumab, bevacizumab, cetuximab, panitumumab, sunitinib, sorafenib, gefitinib, erlotinib, temsirolimus. ado-trastuzumab emtansine, crizotinib, pertuzumab, ramucirumab, regorafenib, vemurafenib, abiraterone acetate, ziv-aflibercept and the like. Examples of immune checkpoint inhibitors include but are not limited to anti-PD-1 antibody, anti-PD-L1 antibody, anti-PDL-2 antibody, anti-CTLA4 antibody.

In some aspects, the present invention provides a method for treating, delaying or preventing the metastases of tumor in a subject comprising administering a therapeutically effective amount of one or more monoclonal antibodies that bind and neutralize both LGALS3BP and PD-1 axis wherein said subject has been diagnosed for tumor associated with increased/altered levels of LGALS3BP and PD-1 axis.

In some aspects, the present invention provides a method for treating, delaying or preventing the metastases of tumor in a subject comprising administering a therapeutically effective amount of one or more monoclonal antibodies that bind and neutralize both LGALS3BP and CTLA4 wherein said subject has been diagnosed for tumor associated with increased/altered levels of LGALS3BP and CTLA4.

In some aspects, the present invention provides a monoclonal antibody that binds and neutralizes LGALS3BP for use in the treatment of a tumor ameliorated by stimulation of an immune response, wherein in said treatment an immune checkpoint protein inhibitor, is co-administered.

In some aspects, the present invention provides a combination therapy for the treatment of tumor or cancer, the said combination comprises:

(a) a monoclonal antibody that binds and/or neutralizes LGALS3BP and

(b) an immune checkpoint inhibitor

In some aspects, the present invention provides a kit comprising

(a) a first composition comprising an anti-LGALS3BP antibody and

(b) a second composition comprising an immune checkpoint inhibitor.

### BRIEF DESCRIPTION OF THE DRAWINGS

FIG. 1. shows that in the presence of anti-LGALS3P antibody the tumoricidal activity of the effector CD8+ T cells and the immune check point inhibitor, anti-PD1 on the colon carcinoma HT-29 cells caused a fivefold increase, as % of apoptotic cell captured.

FIG. 2. shows that the combination of anti-LGALS3P and the immune check point inhibitor, anti-PD1 caused a seven-fold increase in the release of IL-2 from the effector CD8+ T cells in the presence of the colon carcinoma HT-29 cells.

### DETAILED DESCRIPTION OF THE INVENTION

### Abbreviations

As used herein, the following abbreviations have the following meanings:
PD-L1 (Programmed cell death ligand 1)
PD-L2 (Programmed cell death ligand 2)
PD1 (Programmed death 1)
CTLA4 (Cytotoxic T-lymphocyte-associated protein 4)
LGALS3BP Lectin galactoside-binding soluble 3 binding protein
CD (Cluster of differentiation)
IL2 Interleukin 2

Various terms are used throughout the specification and claims. Such terms are to be given their ordinary meaning in the art unless otherwise indicated. Other specifically defined terms are to be construed in a manner consistent with the definition provided herein.

In order to curtail the tumor progression as well as to develop effective therapeutic anti-tumor strategies, key immune regulators have been recognized and are called as immune checkpoints. Immune checkpoints refer to a number of inhibitory players which are involved in immune responses that are crucial for maintaining self-tolerance and modulating the duration and amplitude of physiological immune responses in order to prevent excess tissue damage. Therapies targeting such immune checkpoints such as CTLA-4, PD-1 have shown great success clinically as they boost the already existing immune response working against progressing tumor. The CD33-related subset of sialic acid-binding immunoglobulin-like lectins (Siglecs) consists of similar immunomodulatory molecules that have recently been associated with the modulation of immune responses to cancer. Because up-regulation of Siglec ligands in cancer tissue has been observed, the characterization of these cancer-associated ligands that bind to inhibitory CD33-related Siglecs could provide novel targets for cancer immunomodulatory therapy. Siglecs are a family of sialic-acid-binding immunoglobulin-like lectins that are thought to promote cell-cell interactions and regulate the functions of cells in the innate and adaptive immune systems through glycan recognition. Lectin galactoside-binding soluble 3 binding protein (LGALS3BP, also called Mac-2 binding protein, M2BP, Mac-2BP, BTBD17B, Galectin-3 Binding Protein, 90K) has been identified as a ligand for CD33-related Siglecs.

Lectin galactoside-binding soluble 3 binding protein (LGALS3BP, also called Mac-2 binding protein, 90k protein) is a heavily glycosylated secreted molecule that has been shown previously to be up-regulated in many cancers including breast, NSCLC, colorectal, prostate, pancreatic, colon, ovarian, melanoma, hepatoma, esophageal and gastric, renal, thyroid and urothelial cancer as well as in the extracellular matrix of the cancer associated tissue. It overexpression has also been recorded in the patients infected from the human immunodeficiency virus (HIV). LGALS3BP has been implicated in tumor metastatic processes, as well as in other cell adhesion and immune functions. The upregulation of LGALS3BP has been implicated in influencing T cell activation as well as NK cell response against tumors and thereby hamper the generation of an antitumoral (Thl) immune response. Binding of LGALS3BP to integrins on tumor cells activate the Akt and Raf-Erk pathways, which is associated with increased survival, proliferation, motility, and migration of cancer cell lines. Moreover, it being a ligand of siglec is able to inhibit neutrophil activation in a sialic acid- and Siglec-dependent manner. This indicates that immune cell activation could be modulated via an LGALS3BP-CD33rSiglec pathway during cancer progression and that tumor cells could evade immunosurveillance by up-regulating LGALS3BP. Hence, targeting LGALS3BP could be used as an immune-oncology agent for the treatment of various cancers.

Since many of the immune checkpoints are also regulated by interactions between specific receptor and ligand pairs, monoclonal antibodies or other agents can be used to block this interaction and prevent immunosuppression. The two checkpoint receptors that have received the most attention in recent years are CTLA-4 and PD-1. CTLA-4, PD-1 and its ligands are members of the CD28-B7 family of co-signaling molecules that play important roles throughout all stages of T-cell function and other cell functions. The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and essentially switches it off or inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response.

A first-in-class immunotherapy, ipilimumab (Yervoy®), a monoclonal antibody that targets cytotoxic T-lymphocyte-associated antigen 4 (CTLA-4) on the surface of T cells, was approval for the treatment of melanoma. Now, a new targeted immunotherapy aimed at the programmed death-1 (PD-1) T-cell receptor or its ligand (PD-L1 or PD-L2) may prove to be more effective and even safer than ipilimumab. Additional checkpoint targets may also prove to be effective, such as TIM-3, LAG-3, various B-7 ligands, CHK 1 and CHK2 kinases, BTLA, A2aR, and others.

PDL1 expression in tumors has been associated with poor prognosis in many tumor types, which has been interpreted as consistent with its role in immune evasion. However, recent reports have challenged this notion to some extent, documenting favorable outcomes in melanoma patients with PDL1 positive tumors. PDL1 expression in the tumors was co-localized with tumor T cell infiltration and interferon-γ mRNA expression, suggesting an “adaptive resistance” mechanism in which PDL1 expression is a reflection for the melanoma being actively attacked by presumably melanoma-specific T cells, explaining the improved prognosis. Therefore, the clinical efficacy seen with PD-1/PDL1 pathway blockade in patients with multiple different tumor types, most of whom were heavily pretreated, suggests that the PD-1 pathway is an important target that many tumors may utilize to evade destruction by the host immune response. This observation in conjunction with the favorable toxicity profile of PD-1 inhibition indicates potential broad applicability in patients with advanced tumors. Even more meaningful may be the durability of tumor responses observed with PD-1/PDL1 pathway inhibition, which has reached the 10-year mark for some melanoma patients who have not required any treatment for many years. Currently, at least seven checkpoint inhibitor agents are in clinical trials. Among them are monoclonal anti-PD-1 antibodies, both fully human and humanized, as well as a fully human anti-PD-L1 antibody and a fusion protein combining the extracellular domain of PD-L2 and IgGl. Each of these agents is designed to block the interaction between PD-1 and its ligands, and thus keep the T-cell (or other cell) on/off switch in the “on” position, although they each have slightly different mechanisms of action

The upregulation of PDL1 is a common phenomenon in leukemia, lymphomas and other associated cancers that leads to double T-cell immunodeficiency, low proliferation and activation effects, and higher immune suppression in patients. Likewise, the galectin-binding glycoprotein, LGALS3BP is known to be a paramount contributor to the events associated with tumor growth and metastasis, mainly in homotypic cell aggregation. Its multimeric ring like structure allows it to bridge galectins exposed on the surface of tumor cells, thus favoring the formation of homotypic cell aggregates. LGALS3BP contributes to the neoplastic progression, as several evidences point out to its increased expression in sera and neoplastic tissue from cancer patients tightly correlate with poor prognosis and the occurrence of metastasis. For instance, LGALS3BP has been also found to be up-regulated in human colorectal and prostate cancer specimens, particularly in the extracellular matrix. In addition to factors that contribute to tumor progression the existence of the immune evading molecules that otherwise act as negative regulators limiting the magnitude and duration of the response to prevent healthy tissue damage, have been extensively manipulated by the tumors and their micro microenvironment during development to escape immune detection and eradication. Many human solid tumors express PD ligand 1 (PD-L1), and this is often associated with a worse prognosis. Tumor-infiltrating lymphocytes from patients with cancer typically express PD-1 and have impaired antitumor functionality. Taken together the fact that LGALS3BP is a ligand of inhibitory Siglecs (including Siglec-5 and Siglec-10) expressed predominantly on myeloid cells such as neutrophils and monocytes/macrophages, NK cells (Siglec-7), B-cells, T cells as well as on subpopulation of CD8+ T cells (Siglec10 and Siglec9) the upregulation of LGALS3BP may directly influence T cell activation against tumors and prevent the generation of a strong tumoricidal immune response.

The present invention relates to a combination of anti-LGALS3BP antibody and an immune checkpoint inhibitor promotes an effective anti-tumor response. The details of the various features of the present invention are as follows:

Various antibodies of the present invention are described below:

I. Antibodies

Anti-LGALS3BP antibodies

One or more monoclonal antibodies that bind and/or neutralizes LGALS3BP may include new invented monoclonal antibody against LGALS3BP or well-known antibody such as monoclonal antibody SP-2 (also called as MP-1959). Any anti LGALS3BP antibodies known in the art and described herein may be used in the methods. In some embodiments, the methods, uses, compositions, and kits described herein, the anti-LGALS3BP antibody is SP-2 antibody described herein. The SP-2 was patented as a reagent to determine the concentration of 90K in vitro, for diagnosis and prognosis of patients affected by HIV infection (U.S. Pat. No. 5,298,391). The murine hybridoma cell line from which SP-2 is purified, was deposited by Stefano Iacobelli at the DSMZ (DEUTSCHE SAMMLUNG VON MIKROORGANISMEN UND ZELLKULTUREN GmbH), Mascheroder Weg 1 B D-3300 Braunschweig, Germany under the Budapest Treaty, accession number DSM ACC2116, on Feb. 5, 1993, and at the C.N.C.M. (Collection Nationale de Cultures de Microorganismes), Pasteur Institute of Paris, France, accession number I-1083. SP-2 antibody produced by hybridoma DSM ACC 2116. The DSM ACC 2116 monoclonal antibody SP-2 is produced according to the procedures described by Kohler and Milstein, but it may be produced also according to the recombinant DNA technique, using the specific nucleotide sequence of SP-2 or a part thereof. SP-2 (MP-1959) is a murine monoclonal antibody recognizing LGALS3BP (also known as 90K or Mac-2 BP), a glycoprotein secreted in large amounts by the majority of tumor cells, which plays an important role in cell-cell and cell-extracellular matrix adhesion and invasion. Recently, data have been presented that LGALS3BP functions critically as a pro-angiogenic factor through a dual mechanism, i.e by induction of tumor VEGF and stimulation of endothelial cell tubulogenesis, which is inhibited by SP-2 (Piccolo et al., J Mol Med 91:83-94, 2013)

The antibody SP-2 was generated by immunizing mice with proteins secreted into culture medium of human breast cancer cells (9). Hybridoma cells were cultured in a bench-top BioFlo 3000 bioreactor (New Brunswick Scientific) using serum-free BD Cell MAb Medium (Becton Dickinson). During a three-week growth period, cell proliferation and antibody production were monitored once a week. Medium was collected, centrifuged at 1,200 rpm for 5 minutes to remove cell debris and concentrated using Vivaflow-200 membrane (Sartorius Stedim Biotech). The antibody was purified on a Protein-G column (Biovision) and dialyzed against PBS. The antibody was found about 95% pure, as judged by Coomassie blue staining of SDS-PAGE.

The anti-LGALS3BP antibody is able to bind or neutralize LGALS3BP or 90K protein or 90K said antibody being able to recognize a conformational epitope between residues 107 and 435 of the amino acid sequence of the 90K protein. According to the invention, the antibody may be a human antibody, a humanized antibody, bi-specific antibody or a chimeric antibody. Moreover, the antibody may consist of Fab, Fab′2, scFv, SMIP, affibody, avimer, nanobody or “domain antibody”. Anti LGALS3BP antibody includes antibodies which are raised in mouse using a human recombinant protein fragment corresponding to amino acids 19-300 of human LGALS3BP (NP_005558) produced in E. coli as the immunogen. Other anti-LGALS3BP monoclonal antibodies available included those produced by hybridoma 1A4.21, 2A9.44 and 3C12 [27] and the antibody 2A9.41 that is a subclone of 2A9.44. In other aspects, the anti-LGALS3BP may be a nanobody. Nanobody technology was developed from the discovery that antibodies from camels and llamas (Camelidae, camelids) have heavy chains but no light chains. The antigen-binding site of such antibodies is one single domain, and may be referred to as VHH. See, e.g., U.S. Pat. Nos. 5,800,988 and 6,005,079 and International Application Publication Nos. WO 94/04678 and WO 94/25591, which are incorporated by reference.

Anti-LGALS3BP antibody may be procured, for example, from MyBioSource, Novus Biologicals, OriGene, Atlas Antibodies and Sigma.

Antibodies Interfering with PD-1 Axis

Provided herein is a method for treating or delaying progression of tumor in a subject comprising administering to the subject an effective amount of an antibody interfering with PD-1 axis and an anti-LGALS3BP antibody. For example, the antibodies interfering with PD-1 axis includes an anti PD-1 antibody, an anti-PD-L1 antibody and an anti-PD-L2 antibody. Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the antibodies interfering PD-1 axis is an antibody that inhibits the binding of PD-1 to its ligand binding partners. In a specific aspect the PD-1 ligand binding partners are PD-L1 and/or PD-L2. In another embodiment, an anti-PD-L1 antibody is an antibody that inhibits the binding of PD-L1 to its binding partners. In a specific aspect, PD-L1 binding partners are PD-1 and/or B7-1. In another embodiment, the anti-PD-L2 antibody is an antibody that inhibits the binding of PD-L2 to its binding partners. In a specific aspect, a PD-L2 binding partner is PD-1.

In some embodiment, the antibodies interfering with PD-1 is an anti-PD-1 antibody (e.g., a human antibody, a humanized antibody, or a chimeric antibody). In some embodiments, the anti-PD-1 antibody is selected from the group consisting of MDX-1106 (also known as nivolumab, MDX-1106-04, ONO-4538, BMS-936558, and OPDIVO®), Merck 3475 (also known as pembrolizumab, MK-3475, lambrolizumab, KEYTRUDA®, and SCH-900475), and CT-011 (also known as pidilizumab, hBAT, and hBAT-1. In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55.S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in WO2006/121168. Merck 3745, also known as MK-3475 or SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

In some embodiments, the anti-PD-1 antibody is MDX-1106. Alternative names for “MDX-1106” include MDX-1106-04, ONO-4538, BMS-936558 or nivolumab. In some embodiments, the anti-PD-1 antibody is Nivolumab (CAS Registry Number: 946414-94-4).

In some embodiments, the anti PD-L2 antibody is AMP-224 or rHIgM12B7.

Examples of anti-PD-L1 antibodies useful for the methods of this invention, and methods for making thereof are described in PCT patent application WO 2010/077634 A1, which is incorporated herein by reference.

The anti-PD-L1 antibodies useful in this invention, including compositions containing such antibodies, such as those described in WO 2010/077634 A1 and U.S. Pat. No. 8,217,149, may be used in combination with an anti-LGALS3BP antibody to treat cancer.

The antibody or antigen binding fragment thereof, may be made using methods known in the art, for example, by a process comprising culturing a host cell containing nucleic acid encoding any of the previously described anti-PD-L1, anti-PD-1, or anti-PD-L2 antibodies or antigen-binding fragment in a form suitable for expression, under conditions suitable to produce such antibody or fragment, and recovering the antibody or fragment.

With regard to anti-PD-1 antibodies, these are known and include nivolumab and lambrolizumab, AMP-224, MDPL3280A, MEDI4736 and MSB0010718C.

Anti-PD-1 antibody may be procured from BPS Biosciences and Bio X cell.

### Anti-CTLA4 Antibodies

Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22 (145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

A preferred clinical CTLA-4 antibody is human monoclonal antibody 10D1 (also referred to as MDX-O1 O and ipilimumab and available from Medarex, Inc., Bloomsbury, N.J.) is disclosed in WO 01/14424.

With regard to anti-CTLA-4 antibodies, these are known and include tremelimumab and ipilimumab.

II. Methods

The current method of use of an anti-LGALS3BP antibody in combination with the immune check point inhibitor(s) therefore interferes with the metastases, as well as lack of response to chemotherapy. The combination hence strategizes to potentiate immunostimulation by not only inhibiting tumor growth and spread but impacting the negative immune-regulatory pathways in the tumor environment in subjects that have increased/altered expression of LGAL3SBP and immune checkpoint inhibitor.

The present inventors have discovered for the first time that the co-administration of an anti-LGALS3BP antibody and an immune checkpoint inhibitor (e.g., an antibody) effectively inhibits tumor growth synergistically. Accordingly, the present invention provides improved methods for treating subjects with cancer. Specifically, the present invention provides efficacious combination treatment regimens wherein an anti-LGALS3BP antibody is combined with an immune checkpoint inhibitor for the treatment of cancer. The inventors of the present invention however have surprisingly found that the combination of an anti-LGALS3BP antibody together with an immune checkpoint inhibitor does have additional effects on T-cell stimulation in comparison to an anti-LGALS3BP antibody or an immune checkpoint inhibitor alone. The methods of this invention may find use in treating conditions where enhanced immunogenicity is desired such as increasing tumor immunogenicity for the treatment of cancer. A variety of cancers may be treated, or their progression may be delayed, which specifically includes solid tumor/cancer.

In some embodiments of the methods, uses, compositions, and kits described herein, the cancer is a solid tumor. In some embodiments, the cancer is urogenital cancers (such as prostate cancer, renal cell cancers, bladder cancers), hormone sensitive or hormone refractory prostate cancer, gynecological cancers (such as ovarian cancers, cervical cancers, endometrial cancers), lung cancer, non-small cell lung cancer, small cell lung cancer, gastrointestinal cancers (such as non-metastatic or metastatic colorectal cancers, pancreatic cancer, gastric cancer, oesophageal cancer, hepatocellular cancer, cholangiocellular cancer), head and neck cancer (such as head and neck squamous cell cancer), malignant glioblastoma, malignant mesothelioma, non-metastatic or metastatic breast cancer (such as hormone refractory metastatic breast cancer, triple negative breast cancer), malignant melanoma, melanoma, merkel cell carcinoma or bone and soft tissue sarcomas, oral squamous cell carcinoma, neuroblastoma and the like. The most preferred cancer is pancreatic cancer, colorectal cancer, prostate cancer, breast cancer, triple negative breast cancer, non-small cell lung cancer, ovarian cancer, oral squamous cell carcinoma, lung cancer, hepatocellular carcinoma gastrointestinal cancer, melanoma, lymphoma, neuroblastoma and metastases thereof.

In some embodiments the methods, uses, compositions and kits described herein, the subject is a human. In some embodiments, the subject has cancer or has been diagnosed with cancer. In some embodiments, the subject is suffering from replaced or refractory cancer (such as solid tumor). In some embodiments, the subject is suffering from solid tumor (such as pancreatic, colorectal, triple negative breast cancer, non-small cell lung cancer, oral squamous cell carcinoma, hepatocellular carcinoma, ovarian cancer, neuroblastoma, melanoma) or hematopoietic cancer (non-Hodgkin's lymphoma, leukemia, multiple myeloma). In some embodiments, the subject is suffering from relapsed or refractory or previously untreated solid tumor.

In yet preferred embodiment, the present invention is used to treat the preferably cancers such as breast cancer, non-small cell lung cancer, colorectal cancer, prostate cancer, pancreatic cancer, colon cancer, ovarian cancer, melanoma, hepatoma, esophageal and gastric cancer, renal cancer, thyroid cancer, neuroendocrine, prostate and urothelial cancer.

In some embodiments, the subject has cancer or is at risk of developing cancer. In some embodiments, the treatment results in a sustained response in the subject after cessation of the treatment. In some embodiments, the subject has cancer that may be at early stage or late stage. In some embodiments, the cancer is metastatic.

The cancers described above can be treated with an anti-LGALS3BP antibody and an immune checkpoint inhibitor, which includes the treatment of LGALS3BP expressing cancer. In some embodiments, the subject treated is suffering from a LGALS3BP expressing cancer. In some embodiments, the cancer has decreased levels of T-cell infiltration. LGALS3BP expression can be used as a biomarker to select patients undergoing said therapy, therefore LGALS3BP would be a marker that would not only guide the expected outcomes of the treatment but also assist in the selection of patients to be appropriately managed at the initial diagnosis to undergo the said therapy.

In one embodiment, the present invention provides a pharmaceutical composition comprising one or more monoclonal antibodies that bind and neutralize both LGALS3BP and an immune checkpoint target and a pharmaceutically acceptable carrier for treating or delaying a tumor/cancer growth or metastases in a subject.

In one embodiment, the present invention provides a pharmaceutical composition comprising one or more monoclonal antibodies that bind and/or neutralize both LGALS3BP and PD 1 axis and a pharmaceutically acceptable carrier for treating or delaying a tumor/cancer growth or metastases in a subject wherein PD-1 axis includes PD-1, PD-L1, PD-L2.

In one embodiment, the present invention provides a pharmaceutical composition comprising one or more monoclonal antibodies that bind and/or neutralize both LGALS3BP and CTLA4 and a pharmaceutically acceptable carrier for treating or delaying a tumor/cancer growth or metastases in a subject.

In one embodiment, the present invention discloses a method of treating or delaying or preventing tumor or cancer in a subject comprising administering to a subject a therapeutically effective amount of an anti-LGALS3BP antibody and an immune checkpoint inhibitor separately, wherein said subject is diagnosed with tumor or cancer.

In one another embodiment, the present invention discloses a method of enhancing, increasing, promoting, modulating desirable immune response in a subject comprising administering to a subject a first composition comprising therapeutically effective amount of an anti-LGALS3BP antibody and a second composition comprising an immune checkpoint inhibitor, wherein said subject is diagnosed with tumor or cancer.

In some embodiments, the present invention provides a monoclonal antibody that binds and neutralizes LGALS3BP for use in the treatment of a tumor ameliorated by stimulation of an immune response, wherein said treatment an immune checkpoint protein inhibitor, is co-administered.

In some embodiments, provided is a method for treating or delaying progression of cancer in a subject comprising administering to the subject an effective amount of an anti-LGALS3BP antibody and an immune checkpoint inhibitor, further comprising administering an additional therapy. The additional therapy may be radiation therapy, surgery (such as lumpectomy and a mastectomy), chemotherapy, gene therapy, DNA therapy, viral therapy, RNA therapy, immunotherapy, bone marrow transplantation, nanotherapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (such as agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. The additional therapy may be one or more of the anti-tumor agents described hereinabove.

In another embodiment, provided herein is use of an anti-LGALS3BP antibody in the manufacture of a pharmaceutical composition for treating or delaying progression of tumor in a subject, wherein the medicament comprises the anti-LGALS3BP antibody and an optional pharmaceutically acceptable carrier, and wherein the treatment comprises administration of the first pharmaceutical composition in combination with a second pharmaceutical composition comprising an immune checkpoint inhibitor and an optional pharmaceutically acceptable carrier.

In another embodiment, provided herein is use of an immune checkpoint inhibitor in the manufacture of a pharmaceutical composition for treating or delaying progression of tumor in a subject, wherein the pharmaceutical composition comprises the immune checkpoint inhibitor and an optional pharmaceutically acceptable carrier, and wherein the treatment comprises administration of the second pharmaceutical composition in combination with a first pharmaceutical composition comprising anti-LGALS3BP antibody and an optional pharmaceutically acceptable carrier.

In another embodiment, provided herein is a first pharmaceutical composition comprising anti-LGALS3BP antibody and an optional pharmaceutically acceptable carrier for use in treating or delaying progression of tumor in a subject, wherein the treatment comprises administration of said first pharmaceutical composition in combination with a second composition, wherein the second composition comprises an immune checkpoint inhibitor and an optional pharmaceutically acceptable carrier.

In another embodiment, provided herein is a second pharmaceutical composition comprising an immune checkpoint inhibitor and an optional pharmaceutically acceptable carrier for use in treating or delaying progression of tumor in a subject, wherein the treatment comprises administration of said second pharmaceutical composition in combination with a first composition, wherein the first composition comprises an anti-LGALS3BP antibody and an optional pharmaceutically acceptable carrier.

In another embodiment, provided herein is use of an anti-LGALS3BP antibody in the manufacture of a first pharmaceutical composition for enhancing immune function in a subject having cancer or tumor, wherein the first pharmaceutical composition comprises the anti-LGALS3BP antibody and an optional pharmaceutically acceptable carrier, and wherein treatment comprises administration of the pharmaceutical composition in combination with a second composition comprising an immune checkpoint inhibitor and an optional pharmaceutically acceptable carrier.

In another embodiment, provided herein is use of an immune checkpoint inhibitor in the manufacture of a pharmaceutical composition for enhancing immune function in a subject having cancer, wherein the second pharmaceutical composition comprises the immune checkpoint inhibitor and an optional pharmaceutically acceptable carrier, and wherein the treatment comprises administration of the second pharmaceutical composition in combination with a first composition comprising an antiLGALS3BP antibody and an optional pharmaceutically acceptable carrier.

In another embodiment, the present invention provides a combination therapy for the treatment of tumor or cancer, the said combination comprises(a) a monoclonal antibody that binds and neutralizes LGALS3BP and (b) an immune checkpoint inhibitor selected from the group comprising of anti PD-1 antibody, anti-PD L-1 antibody, anti PD L-2 antibody or anti-CTLA4 antibody.

In one embodiment, said anti-LGALS3BP antibody is a SP-2 antibody. In other embodiment, said immune checkpoint inhibitors include but are not limited to anti-PD-1 antibody, anti-PD-L1 antibody, anti-PD-L2 antibody, anti-CTLA4 antibody.

In one another embodiment, the present invention discloses a pharmaceutical composition comprising one or more monoclonal antibodies that bind and neutralize LGALS3BP in combination with one or more immune checkpoint inhibitors, along with optional anti-tumor agent(s) and one or more pharmaceutically acceptable excipients and/or adjuvants.

In one another embodiment, the present invention discloses a pharmaceutical composition comprising an anti-LGALS3BP antibody in combination with anti-PD-1 antibody along with optional anti-tumor agent(s) and one or more pharmaceutically acceptable excipients and/or adjuvants.

In one another embodiment, the present invention discloses a pharmaceutical composition comprising an anti-LGALS3BP antibody in combination with anti-PD-1 antibody along with optional anti-tumor agent(s) and one or more pharmaceutically acceptable excipients and/or adjuvants.

In one another embodiment, the present invention discloses a pharmaceutical composition comprising an anti-LGALS3BP antibody in combination with anti-PD-L1 antibody along with optional anti-tumor agent(s) and one or more pharmaceutically acceptable excipients and/or adjuvants.

In one another embodiment, the present invention discloses a pharmaceutical composition comprising an anti-LGALS3BP antibody in combination with anti-PD-L2 antibody along with optional anti-tumor agent(s) and one or more pharmaceutically acceptable excipients and/or adjuvants.

In one another embodiment, the present invention discloses a pharmaceutical composition comprising an anti-LGALS3BP antibody in combination with anti-CTLA4 antibody along with optional anti-tumor agent(s) and one or more pharmaceutically acceptable excipients and/or adjuvants.

The anti-tumor agent may be selected from the group consisting of an antibody, an antimetabolite, a vinca alkaloid, a taxane, an anthracycline, a platin derivative, a small molecule, a kinase inhibitor, an alkylating agent, a mTOR inhibitor. Examples of anti-tumor agents include but not limited: docetaxel, paclitaxel, doxorubicin, farmorubicin, cyclophosphamide, 5-fluorouracil, vinorelbine, cisplatin, carboplatin, trastuzumab, bevacizumab, cetuximab, panitumumab, sunitinib, sorafenib, gefitinib, erlotinib, temsirolimus. Ado-trastuzumab emtansine, crizotinib, pertuzumab, ramucirumab, regorafenib, vemurafenib, abiraterone acetate, ziv-aflibercept and the like.

In one embodiment, the present invention discloses immune response modulation via targeting LGALS3BP-CD33 related Siglec pathway using antibody-molecular targeted therapy or antibody-drug conjugate. The molecular targeted therapies or drug is selected from the group consisting of, but not limited to, trastuzumab, bevacizumab, cetuximab, panitumumab, sunitinib, sorafenib, gefitinib, erlotinib, temsirolimus, ipilimumab, Ado-trastuzumab emtansine, crizotinib, nivolumab, pembrolizumab, pertuzumab, ramucirumab, regorafenib, vemurafenib, abiraterone acetate and Ziv-aflibercept. The word “antibody-molecular targeted therapy” includes combination of anti-LGALS3BP antibody and specific drug or antibody mentioned herein.

In a preferred embodiment, anti-PD1 antibody is selected from group comprising of ANA011, BGB-A317, KD033, pembrolizumab (Keytruda®), MCLA-134, mDX400, MEDI0680, muDX400, nivolumab (Opdivo®), PDR001, PF-06801591, pidilizumab, REGN-2810, SHR-1210, STI-A1110, TSR-042, ANB011, 244C8, 388D4, and TSR042. Preferred antibodies are pembrolizumab, nivolumab or pidilizumab.

In a preferred embodiment, anti-PD L1 antibody is selected from group comprising of avelumab, BMS-936559, durvalumab, MCLA-145, SP142, STI-A1011, STI-A1012, STI-A1010, STI-A1014, A110, KY1003 and atezolimumab and the preferred one is durvalumab or atezolimumab and said anti-PD-L2 antibody is selected from AMP-224 or rHIgM12B7.

In a preferred embodiment, anti-CTLA4 antibody is selected from group comprising of KAHR-102, AGEN1884, ABR002, KN044, tremelimumab or ipilimumab and the preferred one is tremelimumab and ipilimumab.

III. Administration

Suitable administration/treatment protocols for treating cancer or tumor in a subject include, for example, administering to the patient an effective amount of an anti-LGALS3BP antibody and an immune checkpoint inhibitor.

In some embodiments, the combination therapy of the invention comprises administration of an anti-LGALS3BP antibody and an immune checkpoint inhibitor. The anti-LGALS3BP antibody and the immune checkpoint inhibitor may be administered in any suitable manner known in the art. For example, the anti-LGALS3BP antibody and the immune checkpoint inhibitor may be administered sequentially (at different times) or concurrently (at the same time).

In some embodiments, the immune checkpoint inhibitor is administered before administration of the anti-LGALS3BP antibody. In some embodiments, the immune checkpoint inhibitor is administered simultaneously with administration of the anti-LGALS3BP antibody. In some embodiments, the immune checkpoint inhibitor is administered after administration of the anti-LGALS3BP antibody.

In some embodiments, the anti-LGALS3BP antibody or an immune checkpoint inhibitor is administered continuously. In some embodiments, the anti-LGALS3BP antibody or immune checkpoint inhibitor is administered intermittently.

In some embodiments, the immune checkpoint inhibitor and the anti-LGALS3BP antibody is co-administered, for example, the administration of said immune checkpoint inhibitor and the anti-LGALS3BP antibody as two separate formulations. The co-administration can be simultaneous or sequential in either order. In one further embodiment, there is a time period while both (or all) antibodies simultaneously exert their biological activities. Said immune checkpoint inhibitor and said anti-LGALS3BP antibody are co-administered either simultaneously or sequentially (for example, via an intravenous (i.v.) through a continuous infusion. When both antibodies are co-administered sequentially the antibodies are administered in two separate administrations that are separated by a “specific period of time”. The term specific period of time is meant anywhere from 1 hour to 15 days. For example, one of the agents can be administered within about 15, 14, 13, 12, 11, 10, 9, 8, 7, 6, 5, 4, 3, 2, or 1 day, or 24, 23, 22, 21, 20, 19, 18, 17, 16, 15, 14, 13, 12, 11, 10, 9, 8, 7, 6, 5, 4, 3, 2 or 1 hour from the administration of the other antibody, and, in one embodiment, the specific period time is 10, 9, 8, 7, 6, 5, 4, 3, 2, or 1 day, or 24, 23, 22, 21, 20, 19, 18, 17, 16, 15, 14, 13, 12, 11, 10, 9, 8, 7, 6, 5, 4, 3, 2 or 1 hour. In some embodiments, simultaneous administration means at the same time or within a short period of time, usually less than 1 hour.

A dosing period as used herein is meant a period of time, during which each antibody has been administered at least once. A dosing period is usually about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, and, in one embodiment, 6, 7, 8, 9, 10, 11, 12, 13, or 14 days, for example, 7 or 14 days.

In certain embodiments, multiple (for example, 2, 3, 4, 5, 6, 7, 8, 9, 10 or more) doses of an anti-LGALS3BP antibodies and multiple (for example, 2, 3, 4, 5, 6, 7, 8, 9, 10 or more) doses of an immune checkpoint inhibitors are administered to a subject in need of treatment.

In certain embodiments, the immune checkpoint inhibitor is administered in a dose of 0.01 mg/kg, 0.05 mg/kg, 0.1 mg/kg, 0.2 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg or 20 mg/kg. The dose of this antibody may from about 0.01 mg/kg to 20 mg/kg. In certain embodiments, the checkpoint inhibitor is administered one dose per day, one dose every 2 days, one dose every 3 days, one dose every 4 days, one dose every 5 days, twice, once a week, once every two weeks, or once every month. In certain embodiments, the checkpoint inhibitor is administered as a single dose, in two doses, in three doses, in four doses, in five doses, or in 6 or more doses.

In certain embodiments, the anti-LGALS3BP antibody is administered in a dose of 0.25 μg/kg, 0.50 μg/kg, 0.80 μg/kg, 0.90 μg/kg, 1.0 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.2 mg/kg, 0.3 mg/kg, 0.5 mg/kg, 0.7 mg/kg, 0.83 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or 20 mg/kg. Total daily dose may vary from 10 μg to 10 mg, preferably 50 μg to 5 mg. The dose of this antibody may vary from about 0.25 μg/kg to 20 mg/kg, preferably 0.50 μg/kg to 10 mg/kg. In certain embodiments the dose frequency may vary from once a day to once very month.

An effective amount of the anti-LGALS3BP antibody and the immune checkpoint inhibitor may be administered for prevention or treatment of cancer. The appropriate dosage of the anti-LGALS3BP antibody and/or the immune checkpoint inhibitor may be determined based on the type of disease to be treated, the type of the anti-LGALS3BP antibody and the immune checkpoint inhibitor, the severity and course of the disease, the clinical condition of the subject, the subject's clinical history and response to the treatment, and the discretion of the attending physician.

In some embodiments, a method of treating cancer will be performed even with a low likelihood of success, but which, given the medical history and estimated survival expectancy of a patient, is nevertheless deemed to induce an overall beneficial course of action.

Accordingly, in one embodiment, the dose of the anti-LGALS3BP and immune checkpoint inhibitor is calculated per mg/kg body weight. However, in another embodiment, the dose of the anti-LGALS3BP and/or immune checkpoint inhibitor is a flat fixed dose that is fixed irrespective of the weight of the patient.

The anti-LGALS3BP antibody and the immune checkpoint inhibitor may be administered by the same route of administration or by different routes of administration. In some embodiments, the anti-LGALS3BP antibody is administered intravenously, intramuscularly, subcutaneously, topically, orally, transdermally, intraperitoneally, intraorbitally, by implantation, by inhalation, intrathecally, intraventricularly, or intranasally. In some embodiments, the immune checkpoint inhibitor is administered intravenously, intramuscularly, subcutaneously, topically, orally, transdermally, intraperitoneally, intraorbitally, by implantation, by inhalation, intrathecally, intraventricularly, or intranasally.

In some embodiments, the immune checkpoint inhibitor is an anti-PD-L1 antibody. In some embodiments, the anti-PD-L1 antibody is administered to the subject intravenously at a dose of 1200 mg once every three weeks. In some embodiments, the anti-PD-L1 antibody is administered with an anti-LGALS3BP antibody.

IV. Pharmaceutical Composition/Formulations

Also provided herein are pharmaceutical compositions or formulations comprising an anti LGALS3BP antibody and/or an immune checkpoint inhibitor and a pharmaceutically acceptable carrier.

In one embodiment, the invention provides for a composition comprising an anti-LGALS3BP antibody and at least one pharmaceutically acceptable carrier. In some embodiments, the anti-LGALS3BP antibody administered to the subject is a composition comprising one or more pharmaceutically acceptable carrier. Any of the pharmaceutically acceptable carrier described herein or known in the art may be used.

In a still further embodiment, the invention provides for a composition comprising an immune checkpoint inhibitor such as anti-PD-L1, an anti-PD-1, or an anti-PD-L2 antibody or anti-CTLA4 antibody as provided herein and at least one pharmaceutically acceptable carrier. In some embodiments, the anti-PD-L1, anti-PD-1, or anti-PD-L2 antibody or anti-CTLA4 administered to the subject is a composition comprising one or more pharmaceutically acceptable carrier. Any of the pharmaceutically acceptable carrier described herein or known in the art may be used.

As used herein, the term “pharmaceutical composition” refers to a composition comprising at least one active principle (for example, an anti-LGALS3BP antibody or an immune checkpoint inhibitor) and at least one pharmaceutically acceptable carrier. Pharmaceutically acceptable carriers are well known to the skilled in the art, and usually depend on the chosen route of administration. In some embodiments, the mixture comprises at least one anti-LGALS3BP antibody in an amount that results in an additive or a synergistic effect with the at least one immune checkpoint inhibitor in a subject when both are administered simultaneously (for example, in a single formulation or concurrently as separate formulations). In some embodiments, a first composition comprising anti-LGALS3BP antibody and pharmaceutical acceptable carrier and a second composition comprising an immune checkpoint inhibitor and pharmaceutical acceptable carrier wherein both are present in an amount that results in an additive or a synergistic effect when both are administered sequentially (as a separate formulations) to the subject. In another preferred embodiment, the present combination used for treating, prevention and ameliorating the tumor is administered subcutaneously and intravenously.

Pharmaceutical compositions suitable for administration to human patients are typically formulated for parenteral administration, e.g., in a liquid carrier, or suitable for reconstitution into liquid solution or suspension for parenteral administration. In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. Pharmaceutical compositions and formulations as described herein can be prepared by mixing the antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. The term “carrier” refers to a diluent, adjuvant, excipient, or vehicle with which the compound is administered. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; chelating agents such as EDTA; monosaccharides, disaccharides, and other carbohydrates including sugars such as sucrose, mannitol, trehalose or sorbitol, glucose, mannose, or dextrins; salt-forming counter-ions such as sodium; metal complexes (for example, Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.).

The present invention also provides other formulation such as microcapsules, nanoparticles or sustained release compositions, intranasal compositions, oral compositions. Active agents may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nano-capsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, wherein the matrices are in the form of shaped articles, e.g. films, or microcapsules. The formulations to be used for in vivo administration are generally sterile. Sterility may be readily accomplished, e.g., by filtration through sterile filtration membranes

The amount of anti-LGALS3BP antibody present in a composition should, in general, be in the range of about 0.01 to about 30% w/w and preferably in an amount of 0.5 to 20% w/w of the composition. Similarly, the amount of an immune checkpoint inhibitor present in a composition in the range of about 0.01 to about 30% w/w and preferably in an amount of 0.5 to 20% w/w of the composition. The immune checkpoint inhibitor is selected from the group comprising of anti-PD-1, anti-PD-L1, anti-PD-L2, anti-CTLA4 antibody.

In some embodiments, the anti-PD-L1 antibody described herein is in a formulation comprising the antibody at an amount of about 60 mg/mL, histidine acetate in a concentration of about 20 mM, sucrose in a concentration of about 120 mM, and polysorbate (e.g., polysorbate 20) in a concentration of 0.04 % (w/v), and the formulation has a pH of about 5.8. In some embodiments, the anti-PD-L1 antibody described herein is in a formulation comprising the antibody in an amount of about 125 mg/mL, histidine acetate in a concentration of about 20 mM, sucrose is in a concentration of about 240 mM, and polysorbate (e.g., polysorbate 20) in a concentration of 0.02% (w/v), and the formulation has a pH of about 5.5.

In some embodiments, the anti-LGALS3BP antibody described herein is in a formulation comprising a therapeutically effective amount of antibody, and a pharmaceutically acceptable carrier selected from the group comprising bulking agent, buffer, surfactant, pH modifier and the formulation has an appropriate pH.

Liquid preparations may also include solutions for intranasal administration.

Aerosol preparations suitable for inhalation may include solutions and solids in powder form, which may be in combination with a pharmaceutically acceptable carrier, such as an inert compressed gas.

For oral use, the pharmaceutical compositions of the present invention, may be administered, for example, in the form of tablets or capsules, powders, dispersible granules, or cachets, or as aqueous solutions or suspensions

V. Kits

In another aspect, provided is a kit comprising an anti-LGALS3BP antibody and/or an immune checkpoint inhibitor for treating or delaying progression of a cancer in a subject or for enhancing immune function of a subject having cancer. In some embodiments, the kit comprises an anti-LGALS3BP antibody and a package insert comprising instructions for using the anti-LGALS3BP antibody in combination with an immune checkpoint inhibitor to treat or delay progression of cancer in a subject or to enhance immune function of a subject having cancer. In some embodiments, the kit comprises an immune checkpoint inhibitor and a package insert comprising instructions for using the immune checkpoint inhibitor in combination with an anti-LGALS3BP antibody to treat or delay progression of cancer in a subject or to enhance immune function of a subject having cancer. In some embodiments, the kit comprises an anti-LGALS3BP antibody and an immune checkpoint inhibitor, and a package insert comprising instructions for using the anti-LGALS3BP antibody and the immune checkpoint inhibitor to treat or delay progression of cancer in a subject or to enhance immune function of a subject having cancer. Any of the anti-LGALS3BP antibodies and/or immune checkpoint inhibitors described herein may be included in the kits.

In some embodiments, the kit comprises a container containing one or more of the anti LGALS3BP antibodies and immune checkpoint inhibitors described herein. Suitable containers include, for example, bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container) or a package insert. The label or the package insert may indicate that the compound contained therein may be useful or intended for treating or delaying progression of cancer in a subject or for enhancing immune function of a subject having cancer. The kit may further comprise other materials desirable from a commercial and user standpoint, including other buffers, diluents, filters, needles, and syringes. In one embodiment of the invention, an immune checkpoint inhibitor is anti-PD-1 antibody, anti-PD-L1 antibody, anti-PD-L2 antibody or anti-CTLA4 antibody.

Thus, in some embodiments, the present invention is directed to kits which comprise a first composition comprising the one or more anti-LGALS3BP antibodies, and a second composition comprising one or more immune checkpoint inhibitors. In some embodiments, the first and second composition may be mixed together before administering to a subject. In some embodiments, the first and second compositions, may be administered either simultaneously or sequentially (i.e., spaced out over a period of time) so as to obtain the maximum efficacy, additivity, synergy, or a combination thereof of the combination.).

The dosage regimen of the active principles and of the pharmaceutical composition described herein can be chosen by prescribing physicians, based on their knowledge of the art, including information published by regulatory authorities. For example, Nivolumab (Opdivo®) is typically administered intravenously. According to the U.S. Food and Drug Administration (FDA), the recommended dose of OPDIVO® is 3 mg/kg administered as an intravenous infusion over 60 minutes every 2 weeks until disease progression.

In some embodiments of the methods, uses, compositions, and kits described herein, the immune checkpoint inhibitor is selected from the group consisting of an anti-PD-1 antibody, an anti-PD-L1 antibody and an anti PD-L2 antibody. In some embodiments, the PD-1 axis binding antagonist is a -PD-1 binding antagonist. In some embodiments, the anti PD-1 binding antagonist inhibits the binding of PD-1 to its ligand binding partners. In some embodiments, the anti-PD-1 antibody inhibits the binding of PD-1 to PD-L1, PD-1 to PD-L2, or PD-1 to both PD-L1 and PD-L2.

VI. Outcomes

In one embodiment, the treatment produces at least one therapeutic effect selected from the group consisting of reduction in size of a tumor, reduction in a number of metastatic lesions over time, complete response, partial response and stable disease. In yet another embodiment, one or more of the following can occur: the number of cancer cells can be reduced, tumor size can be reduced, cancer cell infiltration into peripheral organs can be inhibited, retarded, slowed or stopped; tumor metastases can be inhibited or slowed, tumor growth can be inhibited, apoptosis measure, Interleukin-2 expression.

In another embodiment, administration of an anti-LGALS3BP antibody and an immune checkpoint inhibitor results in at least a three-fold reduction (e.g., a 3.5-fold reduction) in tumor volume, e.g., relative to treatment with the anti-LGALS3BP antibody or the immune checkpoint inhibitor alone or relative to tumor growth on the first day of treatment or immediately before initiation of treatment.

In another embodiment, administration of an anti-LGALS3BP antibody and an immune checkpoint inhibitor results in at least a three-fold increase (e.g., a 3.5-fold reduction) in % apoptotic cell captured, e.g., relative to treatment with the anti-LGALS3BP antibody or the immune checkpoint inhibitor alone

In a further embodiment, administration of an anti-LGALS3BP antibody and an immune checkpoint inhibitor results in tumor growth inhibition of at least 80%, e.g., relative to treatment with the anti-LGALS3BP antibody or an immune checkpoint inhibitor alone or relative to tumor growth on the first day of treatment or immediately before initiation of treatment.

In certain embodiments, administration of an anti-LGALS3BP antibody and an immune checkpoint inhibitor reduces tumor mass by at least 10%, 20%, 30%, 40%, 50%, 60%, 70%, 80%, 90%, 95%, 99% relative to the tumor mass prior to initiation of the treatment or on the first day of treatment. In some embodiment, the tumor mass is no longer detectable following treatment as described herein. In some embodiments, a subject is in partial or full remission.

In one embodiment, the combination therapy of the present invention is being tested in the mouse model of colorectal cancer Those of skill in the art should, in light of the present disclosure, appreciate that many changes or variations can be made in the specific embodiments which are disclosed and still obtain a like or similar result without departing from the spirit and scope of the invention. The present invention is not to be limited in scope by the specific embodiments described herein (which are intended only as illustrations of aspects of the invention), and functionally equivalent methods and components are within the scope of the invention. Indeed, various modifications of the invention, in addition to those shown and described herein, will become apparent to those skilled in the art from the foregoing description.

The following examples are provided to further illustrate the embodiments of the present invention, but are not intended to limit the scope of the invention. While they are typical of those that might be used, other procedures, methodologies, or techniques known to those skilled in the art may alternatively be used.

The following embodiments further describe the objects of the present invention in accordance with the best mode of practice, however, disclosed invention is not restricted to the particular embodiments hereinafter described.

VII. EMBODIMENTS:

Embodiment 1. A method of enhancing, increasing, promoting, expressing, modulating desirable immune response in a subject, comprising administering an antibody(s) targeting tumors associated with increased level of Lectin galactoside-binding soluble 3 binding protein (LGALS3BP) in an amount to enhance, increase, promote, express, modulate immune response in the subject, wherein the subject has been diagnosed for cancer/tumor.

Embodiment 2. A method of using anti LGALS3BP monoclonal antibodies for enhancing, increasing, promoting, expressing, modulating desirable immune response for prevention and/or treatment of tumors and metastases thereof.

Embodiment 3. A method of enhancing, increasing, promoting, expressing, modulating desirable immune response in a subject, comprising administering a therapeutically effective amount of one or more monoclonal antibodies that bind and neutralize both LGALS3BP and an immune checkpoint target to enhance, increase, promote, express, modulate immune response(s) in the subject, wherein said subject has been diagnosed for tumor associated with increased/altered levels of LGALS3BP and/or immune checkpoint(s).

Embodiment 4. The method according to embodiment 3, wherein said immune checkpoint target is selected from PD1, PDL1, PDL2, CTLA4.

Embodiment 5. The method according to embodiment 3, wherein monoclonal antibody that binds and neutralizes LGALS3BP is anti-LGALS3BP antibody.

Embodiment 6. The method according to embodiment 5, wherein the said anti-LGALS3BP antibody is SP-2 antibody.

Embodiment 7. The method according to embodiment 3, wherein monoclonal antibody (immune checkpoint inhibitor) that binds and neutralizes an immune checkpoint target is selected from anti-PD-1 antibody, anti-PD-L1 antibody, anti-PD-L2 antibody, anti-CTLA4 antibody and combination thereof.

Embodiment 8. The method according to embodiment 7, wherein said anti-PD-1 antibody is selected from the group comprising of ANA011, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 and the preferred one is pembrolizumab, nivolumab or pidilizumab.

Embodiment 9. The method according to embodiment 7, wherein anti-PD-L1 antibody is selected from the group comprising of avelumab, BMS-936559, durvalumab, MCLA-145, SP142, STI-A1011, STI-A1012, STI-A1010, STI-A1014, A110, KY1003 and atezolimumab and the preferred one is durvalumab or atezolimumab and said anti-PD-L2 antibody is selected from AMP-224 or rHIgM12B7.

Embodiment 10. The method according to embodiment 7, wherein anti-CTLA4 antibody is selected from the group comprising of KAHR-102, AGEN1884, ABR002, KN044, tremelimumab or ipilimumab and the preferred one is tremelimumab and ipilimumab.

Embodiment 11. The method according to embodiment 1 or 3, wherein tumor/cancer is selected from the group comprising of pancreatic cancer, colorectal cancer, prostate cancer, breast cancer, triple negative breast cancer, non -small cell lung cancer, ovarian cancer, oral squamous cell carcinoma, lung cancer, hepatocellular carcinoma, gastrointestinal cancer, melanoma, lymphoma, neuroblastoma and metastases thereof.

Embodiment 12. A pharmaceutical composition comprising

(a) one or more monoclonal antibodies that bind and neutralize both LGALS3BP and an immune checkpoint target

(b) a pharmaceutically acceptable carrier for treating, preventing or delaying a tumor growth or metastases in a subject.

Embodiment 13. The method according to embodiment 12, wherein said immune checkpoint target is selected from PD1, PDL1, PDL2, CTLA4.

Embodiment 14. The method according to embodiment 12, wherein monoclonal antibody that binds and neutralizes LGALS3BP is an anti-LGALS3BP antibody.

Embodiment 15. The method according to embodiment 14, wherein the anti-LGALS3BP antibody is SP-2 antibody.

Embodiment 16. The method according to embodiment 12, wherein monoclonal antibody that neutralizes and/or binds an immune checkpoint target is selected from anti-PD-1 antibody, anti-PD-L1 antibody, anti-PD-L2 antibody, anti-CTLA4 antibody and combination thereof.

Embodiment 17. The method according to embodiment 16, wherein anti-PD-1 antibody is selected from the group comprising of ANA011, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4 and TSR042 and the preferred one is pembrolizumab, nivolumab or pidilizumab.

Embodiment 18. The method according to embodiment 16, wherein anti-PD-L1 antibody is selected from the group comprising of avelumab, BMS-936559, durvalumab, MCLA-145, SP142, STI-A1011, STI-A1012, STI-A1010, STI-A1014, A110, KY1003 and atezolimumab and the preferred one is durvalumab or atezolimumab and said anti-PD-L2 antibody is selected from AMP-224 or rHIgM12B7.

Embodiment 19. The method according to embodiment 16, wherein anti-CTLA4 antibody is selected from the group comprising of KAHR-102, AGEN1884, ABR002, KN044, tremelimumab or ipilimumab and the preferred one is tremelimumab and ipilimumab.

Embodiment 20. A monoclonal antibody that binds and neutralizes LGALS3BP for use in the treatment of a tumor ameliorated by stimulation of an immune response, wherein in said treatment an immune checkpoint inhibitor, is co-administered.

Embodiment 21. A pharmaceutical composition comprising

(a) a monoclonal antibody that binds and/or neutralizes LGALS3BP

(b) an immune checkpoint inhibitor and

(a) a pharmaceutically acceptable carrier(s)

(b) optionally other anti-tumor agents for treating, preventing or delaying a tumor growth or metastases in a subject.

Embodiment 22. A pharmaceutical composition for use in combination with an immune checkpoint inhibitor comprising anti-PD-1 antibody, anti-PD-L1 antibody, anti-PD-L2 and anti-CTLA4 antibody for treating a cancer, wherein the pharmaceutical composition comprises anti-LGALS3BP antibody with a pharmaceutically acceptable diluent or carrier.

Embodiment 23. A method for treating, delaying or preventing the metastases of tumor in a subject comprising administering a therapeutically effective amount of one or more monoclonal antibodies that bind and neutralize both LGALS3BP and PD-1 axis wherein said subject has been diagnosed for tumor associated with increased/altered levels of LGALS3BP and PD-1 axis.

Embodiment 24. A method for treating, delaying or preventing the metastases of tumor in a subject comprising administering a therapeutically effective amount of one or more monoclonal antibodies that bind and neutralize both LGALS3BP and CTLA4 wherein said subject has been diagnosed for tumor associated with increased/altered levels of LGALS3BP and CTLA4.

Embodiment 25. A combination therapy for the treatment of tumor, the said combination comprises (a) a monoclonal antibody that binds and neutralizes LGALS3BP and (b) an immune checkpoint inhibitor.

Embodiment 26. A kit comprising

(a) a first composition comprising an anti-LGALS3BP antibody and

(b) a second composition comprising an immune checkpoint inhibitor.

Embodiment 27. The method of embodiment 23, wherein the PD-1 axis is selected from the group consisting of a PD-1, a PD-L1 and a PD-L2.

Embodiment 28. The method according to embodiments 20, 21, 23, 24, 25, wherein monoclonal antibody that binds and neutralizes LGALS3BP is anti-LGALS3BP antibody.

Embodiment 29. The method according to according to embodiment 28, wherein the anti-LGALS3BP antibody is SP-2 antibody.

Embodiment 30. The method according to embodiment 23, wherein monoclonal antibody that binds and neutralizes PD axis is selected from anti-PD-1 antibody, anti-PD-L1 antibody, anti-PD-L2 antibody and combination thereof.

Embodiment 31. The method according to embodiments 20, 21, 25 and 26, wherein the immune checkpoint inhibitor is selected from anti-PD-1 antibody, anti-PD-L1 antibody, anti-PD-L2 antibody, anti-CTLA4 and combination thereof.

Embodiment 32. The method according to embodiment 31, wherein said anti-PD-1 antibody is selected from the group comprising of ANA011, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042 and the preferred one is pembrolizumab, nivolumab or pidilizumab.

Embodiment 33. The method according to embodiment 31, wherein said anti-PD-L1 antibody is selected from the group comprising of avelumab, BMS-936559, durvalumab, MCLA-145, SP142, STI-A1011, STI-A1012, STI-A1010, STI-A1014, A110, KY1003 and atezolimumab and the preferred one is durvalumab or atezolimumab and said anti-PD-L2 antibody is selected from AMP-224 or rHIgM12B7.

Embodiment 34. The method according to embodiment 31, wherein said anti-CTLA4 antibody is selected from the group comprising of KARR-102, AGEN1884, ABR002, KN044, tremelimumab or ipilimumab and the preferred one is tremelimumab and ipilimumab.

Embodiment 35. A method of treating a subject receiving an immune checkpoint inhibitor for the treatment of tumor, the improvement comprising administering an effective amount of anti-LGALS3BP antibody to the subject in conjunction with said immune checkpoint inhibitor, wherein the effect is to enhance the anti-tumor effects of said immune checkpoint inhibitor, wherein said immune checkpoint inhibitor is anti-PD-1 antibody, anti-PD-L1 antibody, anti-PD-L2 antibody, anti-CTLA4 antibody.

The proposed combinations of the present invention include but are not limited to:

In one of the embodiment, anti-LGALS3BP antibody is used in combination of anti-PD1 or anti-PDL1 for the treatment of the solid tumor.

In one of the embodiment, anti-LGALS3BP antibody is used in combination of anti-PD1 or anti-PDL1 for the treatment of the hematological cancer. treatment of the solid tumor or hematological cancer.

In one of the embodiment, anti-LGALS3BP antibody is used in combination of Opdivo®/Keytruda®/Yervoy® for the treatment of the solid tumor or hematological cancer.

In one of the embodiment, SP-2 antibody is used in combination with Opdivo®/Keytruda®/Yervoy® Melanoma; Stage IV (metastatic): Prescribed combination is Ipilimumab+Dacarbazine, Temozolomide+Anti-LGALS3BP. For BRAF mutations: prescribed combination is vemurafenib+Trametinib+Dabrafenib+Anti-LGALS3BP.

In one of the embodiment, anti-LGALS3BP antibody is used in combination of anti-PD1 antibody for the treatment of the breast cancer.

In one of the aspect of invention, anti-LGALS3BP antibody is used in combination of anti-PDL1 antibody for the treatment of the breast cancer.

In yet another embodiment, anti-LGALS3BP antibody is used in combination of anti-CTLA4 antibody for the treatment of the breast cancer.

In yet another embodiment, SP-2 antibody is used in combination of Nivolumab for the treatment of the breast cancer.

In yet another embodiment, SP-2 antibody is used in combination of Pembrolizumab for the treatment of the breast cancer.

In yet another embodiment, SP-2 antibody is used in combination of Ipilimumab for the treatment of the breast cancer.

In another embodiment, anti-LGALS3BP antibody is used in combination of anti-PD1 antibody for the treatment of the colorectal cancer.

In one of the embodiment, anti-LGALS3BP antibody is used in combination of anti-PDL1 antibody for the treatment of the colorectal cancer.

In one of the aspect of invention, anti-LGALS3BP antibody is used in combination of anti-CTLA4 antibody for the treatment of the colorectal cancer.

In yet another embodiment, SP-2 antibody is used in combination of Nivolumab for the treatment of the colorectal cancer.

In yet another embodiment, SP-2 antibody is used in combination of Pembrolizumab for the treatment of colorectal cancer.

In yet another embodiment, SP-2 antibody is used in combination of Ipilimumab for the treatment of the colorectal cancer.

In yet another embodiment, anti-LGALS3BP antibody is used in combination of anti-PD1 antibody for the treatment of the non-small cell lung cancer.

In another embodiment, anti-LGALS3BP antibody is used in combination of anti-PDL1 antibody for the treatment of the non-small cell lung cancer.

In one of the embodiment, anti-LGALS3BP antibody is used in combination of anti-CTLA4 antibody for the treatment of the non-small cell lung cancer.

In yet another embodiment, SP-2 antibody is used in combination of Nivolumab for the treatment of the non-small cell lung cancer.

In yet another embodiment, SP-2 antibody is used in combination of Pembrolizumab for the treatment of non-small cell lung cancer.

In yet another embodiment, SP-2 antibody is used in combination of Ipilimumab for the treatment of the non-small cell lung cancer.

In one of the aspect of invention, anti-LGALS3BP antibody is used in combination of anti-PD1 antibody for the treatment of the small cell lung cancer.

In yet another embodiment, anti-LGALS3BP antibody is used in combination of anti-PDL1 antibody for the treatment of the small cell lung cancer.

In another embodiment, anti-LGALS3BP antibody is used in combination of anti-CTLA4 antibody for the treatment of the small cell lung cancer.

In yet another embodiment, SP-2 antibody is used in combination of Nivolumab for the treatment of the small cell lung cancer.

In yet another embodiment, SP-2 antibody is used in combination of Pembrolizumab for the treatment of small cell lung cancer.

In yet another embodiment, SP-2 antibody is used in combination of Ipilimumab for the treatment of the small cell lung cancer.

In one of the embodiment, anti-LGALS3BP antibody is used in combination of anti-PD1 antibody for the treatment of the prostate cancer.

In one of the aspect of invention, anti-LGALS3BP antibody is used in combination of anti-PDL1 antibody for the treatment of the prostate cancer.

In yet another embodiment, anti-LGALS3BP antibody is used in combination of anti-CTLA4 antibody for the treatment of the prostate cancer.

In yet another embodiment, SP-2 antibody is used in combination of Nivolumab for the treatment of the prostate cancer.

In yet another embodiment, SP-2 antibody is used in combination of Pembrolizumab for the treatment of prostate cancer.

In yet another embodiment, SP-2 antibody is used in combination of Ipilimumab for the treatment of the prostate cancer.

In another embodiment, anti-LGALS3BP antibody is used in combination of anti-PD1 antibody for the treatment of the pancreatic cancer.

In one of the embodiment, anti-LGALS3BP antibody is used in combination of anti-PDL1 antibody for the treatment of the pancreatic cancer.

In one of the embodiment of invention, anti-LGALS3BP antibody is used in combination of anti-CTLA4 antibody for the treatment of the pancreatic cancer.

In yet another embodiment, SP-2 antibody is used in combination of Nivolumab for the treatment of the pancreatic cancer.

In yet another embodiment, SP-2 antibody is used in combination of Pembrolizumab for the treatment of pancreatic cancer.

In yet another embodiment, SP-2 antibody is used in combination of Ipilimumab for the treatment of the pancreatic cancer.

In yet another embodiment, anti-LGALS3BP antibody is used in combination of anti-PD1 antibody for the treatment of the colon cancer.

In another embodiment, anti-LGALS3BP antibody is used in combination of anti-PDL1 antibody for the treatment of the colon cancer.

In one of the embodiment, anti-LGALS3BP antibody is used in combination of anti-CTLA4 antibody for the treatment of the colon cancer.

In yet another embodiment, SP-2 antibody is used in combination of Nivolumab for the treatment of the colon cancer.

In yet another embodiment, SP-2 antibody is used in combination of Pembrolizumab for the treatment of colon cancer.

In yet another embodiment, SP-2 antibody is used in combination of Ipilimumab for the treatment of the colon cancer.

In one of the aspect of invention, anti-LGALS3BP antibody is used in combination of anti-PD1 antibody for the treatment of the ovarian cancer.

In yet another embodiment, anti-LGALS3BP antibody is used in combination of anti-PDL1 antibody for the treatment of the ovarian cancer.

In another embodiment, anti-LGALS3BP antibody is used in combination of anti-CTLA4 antibody for the treatment of the ovarian cancer.

In yet another embodiment, SP-2 antibody is used in combination of Nivolumab for the treatment of the ovarian cancer.

In yet another embodiment, SP-2 antibody is used in combination of Pembrolizumab for the treatment of ovarian cancer.

In yet another embodiment, SP-2 antibody is used in combination of Ipilimumab for the treatment of the ovarian cancer.

In one of the embodiment, anti-LGALS3BP antibody is used in combination of anti-PD1 antibody for the treatment of the melanoma.

In one of the aspect of invention, anti-LGALS3BP antibody is used in combination of anti-PDL1 antibody for the treatment of the melanoma.

In yet another embodiment, anti-LGALS3BP antibody is used in combination of anti-CTLA4 antibody for the treatment of the melanoma.

In yet another embodiment, SP-2 antibody is used in combination of Nivolumab for the treatment of the melanoma.

In yet another embodiment, SP-2 antibody is used in combination of Pembrolizumab for the treatment of melanoma.

In yet another embodiment, SP-2 antibody is used in combination of Ipilimumab for the treatment of the melanoma.

In another embodiment, anti-LGALS3BP antibody is used in combination of anti-PD1 antibody for the treatment of the hepatoma.

In one of the embodiment, anti-LGALS3BP antibody is used in combination of anti-PDL1 antibody for the treatment of the hepatoma.

In one of the embodiment of invention, anti-LGALS3BP antibody is used in combination of anti-CTLA4 antibody for the treatment of the hepatoma.

In yet another embodiment, SP-2 antibody is used in combination of Nivolumab for the treatment of the hepatoma.

In yet another embodiment, SP-2 antibody is used in combination of Pembrolizumab for the treatment of hepatoma.

In yet another embodiment, SP-2 antibody is used in combination of Ipilimumab for the treatment of the hepatoma.

In yet another embodiment, anti-LGALS3BP antibody is used in combination of anti-PD1 antibody for the treatment of the esophageal and gastric cancer.

In another embodiment, anti-LGALS3BP antibody is used in combination of anti-PDL1 antibody for the treatment of the esophageal and gastric cancer.

In one of the embodiment, anti-LGALS3BP antibody is used in combination of anti-CTLA4 antibody for the treatment of the esophageal and gastric cancer.

In yet another embodiment, SP-2 antibody is used in combination of Nivolumab for the treatment of the esophageal and gastric cancer.

In yet another embodiment, SP-2 antibody is used in combination of Pembrolizumab for the treatment of esophageal and gastric cancer.

In yet another embodiment, SP-2 antibody is used in combination of Ipilimumab for the treatment of the esophageal and gastric cancer.

In one of the aspect of invention, anti-LGALS3BP antibody is used in combination of anti-PD1 antibody for the treatment of the renal cancer.

In yet another embodiment, anti-LGALS3BP antibody is used in combination of anti-PDL1 antibody for the treatment of the renal cancer.

In another embodiment, anti-LGALS3BP antibody is used in combination of anti-CTLA4 antibody for the treatment of the renal cancer.

In yet another embodiment, SP-2 antibody is used in combination of Nivolumab for the treatment of the renal cancer.

In yet another embodiment, SP-2 antibody is used in combination of Pembrolizumab for the treatment of renal cancer.

In yet another embodiment, SP-2 antibody is used in combination of Ipilimumab for the treatment of the renal cancer.

In one of the embodiment, anti-LGALS3BP antibody is used in combination of anti-PD1 antibody for the treatment of the thyroid cancer.

In one of the aspect of invention, anti-LGALS3BP antibody is used in combination of anti-PDL1 antibody for the treatment of the thyroid cancer.

In yet another embodiment, anti-LGALS3BP antibody is used in combination of anti-CTLA4 antibody for the treatment of the thyroid cancer.

In yet another embodiment, SP-2 antibody is used in combination of Nivolumab for the treatment of the thyroid cancer.

In yet another embodiment, SP-2 antibody is used in combination of Pembrolizumab for the treatment of thyroid cancer.

In yet another embodiment, SP-2 antibody is used in combination of Ipilimumab for the treatment of the thyroid cancer.

In another embodiment, anti-LGALS3BP antibody is used in combination of anti-PD1 antibody for the treatment of the urothelial cancer.

In one of the embodiment, anti-LGALS3BP antibody is used in combination of anti-PDL1 antibody for the treatment of the urothelial cancer.

In yet another embodiment, anti-LGALS3BP antibody is used in combination of anti-CTLA4 antibody for the treatment of the urothelial cancer.

In yet another embodiment, SP-2 antibody is used in combination of Nivolumab for the treatment of the urothelial cancer.

In yet another embodiment, SP-2 antibody is used in combination of Pembrolizumab for the treatment of urothelial cancer.

In yet another embodiment, SP-2 antibody is used in combination of Ipilimumab for the treatment of the urothelial cancer.

In another embodiment, anti-LGALS3BP antibody is used in combination of anti-PD1 antibody for the treatment of the neuroendocrine cancer.

In one of the aspect of invention, anti-LGALS3BP antibody is used in combination of anti-PDL1 antibody for the treatment of the neuroendocrine cancer.

In one of the embodiment, anti-LGALS3BP antibody is used in combination of anti-CTLA4 antibody for the treatment of the neuroendocrine cancer.

In yet another embodiment, SP-2 antibody is used in combination of Nivolumab for the treatment of the neuroendocrine cancer.

In yet another embodiment, SP-2 antibody is used in combination of Pembrolizumab for the treatment of neuroendocrine cancer.

In yet another embodiment, SP-2 antibody is used in combination of Ipilimumab for the treatment of the neuroendocrine cancer.

In yet another embodiment, anti-LGALS3BP antibody is used in combination of anti-PD1 antibody for the treatment of the hodgkin's lymphoma.

In one of the of invention, anti-LGALS3BP antibody is used in combination of anti-PDL1 antibody for the treatment of the hodgkin's lymphoma.

In one of the embodiment, anti-LGALS3BP antibody is used in combination of anti-CTLA4 antibody for the treatment of the hodgkin's lymphoma.

In yet another embodiment, SP-2 antibody is used in combination of Nivolumab for the treatment of the hodgkin's lymphoma.

In yet another embodiment, SP-2 antibody is used in combination of Pembrolizumab for the treatment of hodgkin's lymphoma.

In yet another embodiment, SP-2 antibody is used in combination of Ipilimumab for the treatment of the hodgkin's lymphoma.

In yet another embodiment, anti-LGALS3BP antibody is used in combination of anti-PD1 antibody for the treatment of the neuroblastoma.

In one of the of invention, anti-LGALS3BP antibody is used in combination of anti-PDL1 antibody for the treatment of the neuroblastoma.

In one of the embodiment, anti-LGALS3BP antibody is used in combination of anti-CTLA4 antibody for the treatment of the neuroblastoma.

In yet another embodiment, SP-2 antibody is used in combination of Nivolumab for the treatment of the neuroblastoma.

In yet another embodiment, SP-2 antibody is used in combination of Pembrolizumab for the treatment of the neuroblastoma.

In yet another embodiment, SP-2 antibody is used in combination of Ipilimumab for the treatment of the neuroblastoma.

### DEFINITIONS

The term “subject” includes any organism, preferably an animal, more preferably a mammal (e.g., rat, mouse, dog, cat, rabbit) and most preferably a human.

As used herein the term “cancer” can be used interchangeably with “tumor”. The term “cancer” refers to the cancers of wide variety of types, including both solid tumors and non-solid tumors such as leukemia and lymphoma. Carcinomas, sarcomas, myelomas, lymphomas, and leukemia can all be treated using the present invention, including those cancers which have a mixed type. The present invention can be used to treat either malignant or benign tumors. In certain embodiments, the cancer treated is colorectal cancer, osteosarcoma, pancreatic cancer, prostate cancer, head and neck cancer, stomach cancer, renal cancer, cervical cancer, liver cancer, breast cancer, ovarian cancer, bladder cancer, urogenital cancer, fibrosarcoma, bone and connective tissue sarcomas, giant cell carcinoma, squamous cell carcinoma, glioma, adenocarcinoma, clear cell kidney cancer, hemangiosarcoma, kaposi's sarcoma, abdominal cancer, kidney cancer, melanoma, colon cancer, gastric cancer, hematological malignancies, non-small cell lung cancer, neuroblastoma, melanoma and so on.

The term “Treating” within the context of the present invention, means an alleviation of symptoms associated with a disorder or disease, or halt of further progression or worsening of those symptoms, or prevention or prophylaxis of the disease or disorder. For example, within the context of treating patients in relation to the anti-LGALS3BP antibody and an immune checkpoint inhibitor, successful treatment may include a reduction in tumor adhesion and anchorage; an alleviation of symptoms related to a cancerous growth or tumor, or proliferation of diseased tissue; a halting in the progression of a disease such as cancer or in the growth of cancerous cells. Treatment may also include administering the pharmaceutical formulations of an anti-LGALS3BP antibody in combination with an immune checkpoint inhibitor. It may be administered before, during, or after surgical procedure and/or radiation therapy. According to this invention, an anti-LGALS3BP antibody and an immune checkpoint inhibitor can be co-administered into a human subject, the daily dosage will normally be determined by the prescribing physician with the dosage generally varying according to the age, weight, and response of the individual patient, as well as the severity of the patient's symptoms.

When introducing elements disclosed herein, the articles “a”, “an”, “the”, and “said” are intended to mean that there are one or more of the elements.

As used herein the term “effective amount” can be used interchangeably with “therapeutically effective dose,” or “therapeutically effective amount,” and it refers to an amount sufficient to produce the desired effect.

As used herein “pharmaceutical acceptable carrier” refers to a carrier medium which does not interfere with the effectiveness of the biological activity of the active ingredients and which is not toxic to the patient or subject.

The term “pharmaceutical composition” as used in accordance with the present invention relates to compositions that can be formulated in any conventional manner using one or more pharmaceutically acceptable carriers or excipients.

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody.RTM., nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” or “monoclonal antibody composition,” as used herein, refers to an antibody or a composition of antibodies that displays a single binding specificity and affinity for a particular epitope. Accordingly, the term “human monoclonal antibody” or “monoclonal antibody composition” refers to an antibody or a composition of antibodies which displays a single binding specificity and which has variable and optional constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell. The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

As used herein, the term “synergy” refers generally to obtaining a combined effect that is greater the sum of two separate effects. As used herein, the terms “therapeutic synergy”, and “synergistic effect,” when placed in a therapeutic context, refer to a phenomenon where treatment of patients with a combination of therapeutic agents (e.g., anti-LGALS3BP antibody in combination with anti-PD1 or anti-PD L1 or anti CTLA4 ) manifests a therapeutically superior outcome to the outcome achieved by each individual constituent of the combination used at its optimum dose (see, e.g., T. H. Corbett et al., 1982, Cancer Treatment Reports, 66, 1187). In this context a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone.

### EXAMPLE 1

Evaluation of anti-LGAL3SBP Antibody in combination with anti-PD1 on the growth and proliferation of colon carcinoma cells in mixed cultures with hPBMCs or human CD8+ T cells

Materials and methods: Human colorectal adenocarcinoma cell line HT29 were purchased from the American Type Culture Collection (ATCC), DMEM (Invitrogen), RPMI (Thermo Fisher), Heat inactivated FBS (Sigma), 2 mM L-glutamine (Invitrogen), 100 units/ml each penicillin and streptomycin (Invitrogen), Trypsin (Lonza), anti-LGALS3BP (Sigma), anti-PD1 (BPS Bioscience), MACS Seperator, FITC-Annexin V (BD Biosciences), PI (BD Biosciences), 24-well flat bottom plate (Falcon), FACSVerse,

Experimental procedure: HT-29 cells were maintained in DMEM (Invitrogen) supplemented with 10% FBS (Sigma), 2 mM L-glutamine, and 100 units/ml each penicillin and streptomycin (Invitrogen. Cells were grown at 37° C. in the presence of 5% CO2 and were split by trypsinization when they reached ˜80-90% confluence. Human PBMCs collected from the healthy volunteers was used for the isolation of the CD8+ T Cells. The CD8+ T-cells was isolated by depletion of non-target cells. The magnetically labeled non-target cells were depleted by retaining them within a MACS® Column in the magnetic field of a MACS Separator, while the unlabeled CD8+ T cells run through the column. Cells were counted and re-suspended in complete RPMI-1640 at 106/ml. A titration of cell densities (2-3×10̂6 cells/mL to 10̂5 cells/mL) for CD8+ T-cells and HT-29 cells was carried out to get optimal ratio of effector to target cells for the studies. 200 μL of the cell suspension was added to each well of the 24-well flat bottom plate and placed in a humidified 37° C., 5% CO2 incubator in presence or absence of anti-LGAL3SBP (160 ng/ml), with or without the addition of anti-PD1 (1 μg/ml). The CD8+ T cells and HT-29 cell were incubated at varying effector target cell ratios for 48 hours incubation time points in the presence or absence of the said antibodies. After treatment, the media was removed, cells were washed once with 100 μL of complete DMEM, and the cell apoptosis was being assessed by Annexin V/PI staining of culture on FACSVerse.

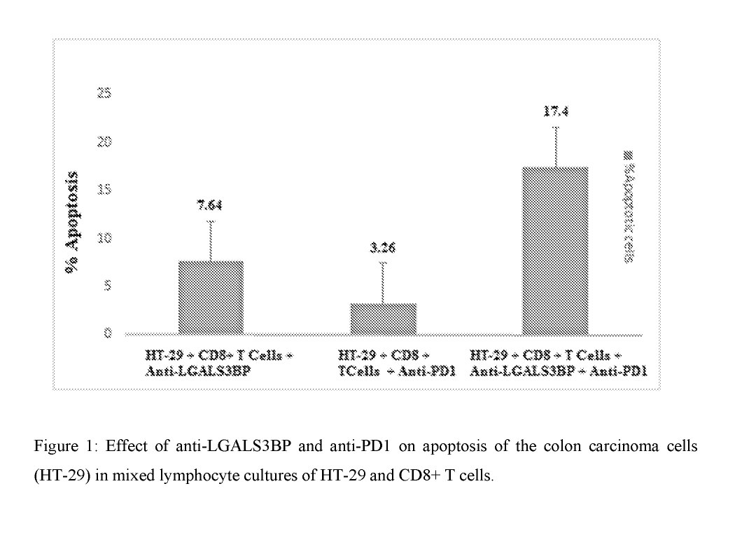

Conclusion: Anti-LGALS3BP at a concentration of 160 ng/ml after 48 hours of incubation at an optimal target to effector cell ratio showed a 7.6% of the HT-29 cells to be apoptotic while anti-PD1 at a concentration of 1 μg/ml caused 3.26% of apoptosis of these carcinoma cells. On the other hand, the combination of anti-LGALS3BP and anti-PD1 at the said concentrations showed a synergistic effect of 17.4% inhibition on the growth of the colon carcinoma cell line as shown in FIG. 1.

### EXAMPLE 2

Evaluation of anti-LGAL3SBP Antibody in combination with anti-PD1 on IL-2 generation in HT-29 colon carcinoma cells cultures with hPBMCs or human CD8+ T cells

Materials and methods: Human colorectal adenocarcinoma cell line HT29 were purchased from the American Type Culture Collection (ATCC), DMEM (Invitrogen), RPMI (Thermo Fisher), Heat inactivated FBS (Sigma), 2 mM L-glutamine (Invitrogen), 100 units/ml each penicillin and streptomycin (Invitrogen), Trypsin (Lonza), anti-LGALS3BP, anti-PD1, MACS Seperator (Milteney), 96-well Immunosorb ELISA plates (Nunc), FACSVerse, IL-2 ELISA kit was procured from ThermoFisher

Experimental procedure: HT-29 cells were maintained in DMEM (Invitrogen) supplemented with 10% FBS (Sigma), 2 mM L-glutamine, and 100 units/ml each penicillin and streptomycin (Invitrogen. Cells were grown at 37° C. in the presence of 5% CO2 and were split by trypsinization when they reached ˜80-90% confluence. Human PBMCs collected from the healthy volunteers was used for the isolation of the CD8+ T Cells. The CD8+ T-cells was isolated by depletion of non-target cells. The magnetically labeled non-target cells were depleted by retaining them within a MACS® Column in the magnetic field of a MACS Separator, while the unlabeled CD8+ T cells run through the column. Cells were counted and re-suspended in complete RPMI-1640 at 106/ml. A titration of cell densities (2-3×10̂6 cells/mL to 10̂5 cells/mL) for CD8+ T-cells and HT-29 cells was carried out to get optimal ratio of effector to target cells for the studies. 200 μL of the cell suspension was added to each well of the 24-well flat bottom plate and placed in a humidified 37° C., 5% CO2 incubator in presence or absence of anti-LGAL3SBP (160 ng/ml), with or without the addition of anti-PD1 (1 μg/ml). The HT-29 cell and CD8+ T cells were incubated at defined cell density ratios in the presence of the said concentrations of the antibodies for 48 hours. After treatment, the supernatant from respective well were collected for the IL-2 estimation. The IL-2 generation was analyzed and measured using the commercially available ELISA (enzyme linked immunosorbent assay) kit as per the manufacturer's protocol.

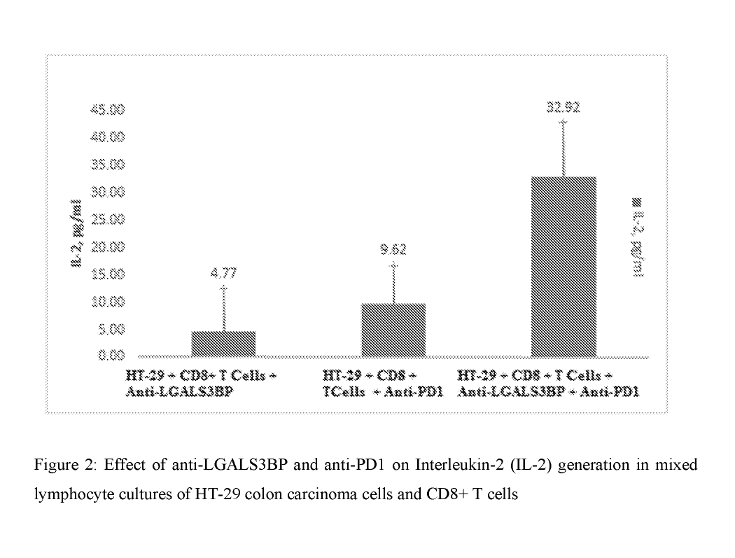

Conclusion: Anti-LGALS3BP at a concentration of 160 ng/ml after 48 hours of incubation at an optimal target to effector cell ratio showed 4.8 pg/ml of IL-2 generation in the cultures of HT-29 cells while anti-PD1 at a concentration of 1 μg/ml caused 9.6 pg/ml of IL-2 release from these carcinoma cells. On the other hand, the combination of anti-LGALS3BP and anti-PD1 at the said concentrations showed a synergistic effect of 32.9 pg/ml of IL-2 generation in the mixed cultures of the colon carcinoma cell line and CD8+T cells as shown in FIG. 2.

Thus it can be concluded that a combination of an anti-LGALS3BP antibody and an immune checkpoint inhibitor produce a synergistic effect.

1. A method of enhancing an immune response and/or treating the metastases of tumor in a subject, comprising administering a therapeutically effective amount of one or more monoclonal antibodies that bind and neutralize both lectin galactoside binding soluble 3 binding protein (LGALS3BP) and an immune checkpoint target.

2. The method according to claim 1, wherein said immune checkpoint target is selected from the group consisting of PD1, PD-L1, PD-L2 and CTLA4.

3. The method according to claim 1, wherein the subject has been diagnosed as having a tumor with increased LGALS3BP levels.

4. The method according to claim 1, wherein the anti-LGALS3BP monoclonal antibody is selected from the group consisting of an SP-2 antibody, an anti-LGALS3BP nanobody, and combinations thereof.

5. The method according to claim 1, wherein said monoclonal antibody of an immune checkpoint target is selected from the group consisting of an anti-PD-1 antibody, an anti-PD-L1 antibody, an anti-PD-L2 antibody, an anti-CTLA4 antibody, and combinations thereof.

6. The method according to claim 5, wherein said anti-PD-1 antibody is selected from the group consisting of ANA011, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042, and combinations thereof.

7. The method according to claim 5, wherein said anti-PD-L1 antibody is selected from the group consisting of avelumab, BMS-936559, durvalumab, MCLA-145, SP142, STI-A1011, STI-A1012, STI-A1010, STI-A1014, A110, KY1003, atezolimumab and, combinations thereof, wherein the preferred one is durvalumab or atezolimumab and wherein said anti-PD-L2 antibody is selected from AMP-224 and rHIgM12B7.

8. The method according to claim 5, wherein said anti-CTLA4 antibody is selected from the group consisting of KAHR-102, AGEN1884, ABR002, KN044, tremelimumab ipilimumab, and combinations thereof, and wherein the preferred one is tremelimumab and ipilimumab.

9. The method according to claim 1, wherein the subject has a cancer selected from the group consisting of pancreatic cancer, colorectal cancer, prostate cancer, breast cancer, triple negative breast cancer, non-small cell lung cancer, ovarian cancer, oral squamous cell carcinoma, lung cancer, hepatocellular carcinoma, gastrointestinal cancer, melanoma, lymphoma, and neuroblastoma.

10. The method of claim 9, wherein the subject has metastatic cancer.

11. -24. (canceled).

25. A combination therapy for the treatment of tumor, said combination therapy comprising (a) a monoclonal antibody that binds and neutralizes LGALS3BP and (b) an immune checkpoint inhibitor.

26-27. (canceled).

28. The combination therapy according to claim 25, wherein said monoclonal antibody that of LGALS3BP is selected from the group consisting of an anti-LGALS3BP nanobody, an SP-2 antibody, and combinations thereof.

29-30. (canceled).

31. The combination therapy according to claim 25, wherein the immune checkpoint inhibitor is selected from the group consisting of an anti-PD-1 antibody, an anti-PD-L1 antibody, an anti-PD-L2 antibody, an anti-CTLA4 antibody, and combinations thereof.

32. The combination therapy according to claim 31, wherein said anti-PD-1 antibody is selected from the group consisting of ANA011, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042, and combinations thereof, and wherein the preferred one is pembrolizumab, nivolumab and pidilizumab.

33. The combination therapy according to claim 31, wherein said anti-PD-L1 antibody is selected from the group consisting f avelumab, BMS-936559, durvalumab, MCLA-145, SP142, STI-A1011, STI-A1012, STI-A1010, STI-A1014, A110, KY1003, atezolimumab, and combinations thereof, and wherein the preferred one is durvalumab and atezolimumab and wherein said anti-PD-L2 antibody is selected from the group consisting of AMP-224, rHIgM12B7, and combinations thereof.

34. The combination therapy according to claim 31, wherein said anti-CTLA4 antibody is selected from the group consisting of KAHR-102, AGEN1884, ABR002, KN044, tremelimumab, ipilimumab, and combinations thereof, and wherein the preferred one is tremelimumab and ipilimumab.

35. (canceled).

36. A method of enhancing IL-2 production in a human having cancer, comprising administering therapeutically effective amounts of (i) an antibody against LGALS3BP and an (ii) immune checkpoint inhibitor to a human having a cancer, wherein the combination of the antibody against LGALS3BP and the immune checkpoint inhibitor provide a synergistic increase in IL-2 production, wherein said immune checkpoint inhibitor is selected from the group consisting of an anti-PD-1 antibody, an anti-PD-L1 antibody, an anti-PD-L2 antibody, an anti-CTLA4 antibody, and combinations thereof.

37. (canceled).

38. The method according to claim 36, wherein said anti-PD-1 antibody is selected from the group consisting of ANA011, BGB-A317, KD033, pembrolizumab, MCLA-134, mDX400, MEDI0680, muDX400, nivolumab, PDR001, PF-06801591, pidilizumab, REGN-2810, SHR-1210, STI-A1110, TSR-042, ANB011, 244C8, 388D4, TSR042, and combinations thereof, and wherein the preferred one is pembrolizumab, nivolumab and pidilizumab.

39. The method according to claim 36, wherein said anti-PD-L1 antibody is selected from the group consisting of avelumab, BMS-936559, durvalumab, MCLA-145, SP142, STI-A1011, STI-A1012, STI-A1010, STI-A1014, A110, KY1003, atezolimumab, and combinations thereof, and the preferred one is durvalumab and atezolimumab, and wherein said anti-PD-L2 antibody is selected from AMP-224 and rHIgM12B7.

40. The combination therapy according to claim 25, wherein the tumor is selected from the group consisting of pancreatic cancer, colorectal cancer, prostate cancer, breast cancer, triple negative breast cancer, non-small cell lung cancer, ovarian cancer, oral squamous cell carcinoma, lung cancer, hepatocellular carcinoma, gastrointestinal cancer, melanoma, lymphoma, and neuroblastoma.

41. The method according to claim 6, wherein said anti-PD-1 antibody is selected from the group consisting of pembrolizumab, nivolumab, and pidilizumab, and combinations thereof.
